(19) United States
(12) Patent Application Publication
(10) Pub. No.: US 2005/0249685 A1
(43) Pub. Date: Nov. 10, 2005

(54) Reduction of hair growth

(76) Inventors: Natalia Botchkareva, Sharon, MA (US); Gurpreet S. Ahluwalia, Wellesley, MA (US); Douglas Shander, Acton, MA (US)

Correspondence Address: FISH & RICHARDSON PC, P.O. BOX 1022, MINNEAPOLIS, MN, 55440-1022, US

(21) Appl. No.: 10/833,673

(22) Filed: Apr. 27, 2004

(51) Int. Cl. A61K 7/06; A61K 31/325

(52) U.S. Cl. 424/70.1; 514/183; 514/476

(57) ABSTRACT

Mammalian hair growth is reduced by topically applying a composition including a heat shock protein inhibitor.

### BACKGROUND

The invention relates to reducing hair growth in mammals, particularly for cosmetic purposes.

A main function of mammalian hair is to provide environmental protection. However, that function has largely been lost in humans, in whom hair is kept or removed from various parts of the body essentially for cosmetic reasons. For example, it is generally preferred to have hair on the scalp but not on the face.

Various procedures have been employed to remove unwanted hair, including shaving, electrolysis, depilatory creams or lotions, waxing, plucking, and therapeutic antiandrogens. These conventional procedures generally have drawbacks associated with them. Shaving, for instance, can cause nicks and cuts, and can leave a perception of an increase in the rate of hair regrowth. Shaving also can leave an undesirable stubble. Electrolysis, on the other hand, can keep a treated area free of hair for prolonged periods of time, but can be expensive, painful, and sometimes leaves scarring. Depilatory creams, though very effective, typically are not recommended for frequent use due to their high irritancy potential. Waxing and plucking can cause pain, discomfort, and poor removal of short hair. Finally, antiandrogens—which have been used to treat female hirsutism—can have unwanted side effects.

It has previously been disclosed that the rate and character of hair growth can be altered by applying to the skin inhibitors of certain enzymes. These inhibitors include inhibitors of 5-alpha reductase, ornithine decarboxylase, S-adenosylmethionine decarboxylase, gamma-glutamyl transpeptidase, and transglutaminase. See, for example, Breuer et al., U.S. Pat. No. 4,885,289; Shander, U.S. Pat. No. 4,720,489; Ahluwalia, U.S. Pat. No. 5,095,007; Ahluwalia et al., U.S. Pat. No. 5,096,911; and Shander et al., U.S. Pat. No. 5,132,293.

Heat shock proteins (HSPs) are a known superfamily of evolutionary conserved proteins, which consist of sub-families with different molecular weight. Examples of HSPs include HSP-27, HSP-70, and HSP-90. HSPs perform multiple intracellular functions. They are also called “stress proteins”, because their synthesis is stimulated by variety of stresses, including cytotoxic drugs, heat, and irradiation. HSPs may play a role in maintenance of cellular homeostasis under physiological conditions as well. Synthesis of HSPs occurs as a result of transcriptional activation of responsive elements by heat shock specific transcription factors, inhibition of which leads to decrease in the level of HSPs. HSPs act as chaperone molecules that bind to client proteins to facilitate their proper folding, assist protein transport and sorting between intracellular compartments, and control their switching between active/native conformation. Among the substrates of HSPs are a number of tyrosine, serine/threonine, and cyclin dependent kinases. In addition, HSP-90 is involved in modulating signaling through hormone receptors. Interactions of HSPs with their dependent proteins are required for regulation of cell proliferation and differentiation. In addition to cell cycle regulation, HSPs may protect cells against programmed cell death, referred as apoptosis, induced by wide variety of stimuli. HSPs possess substantial anti-apoptotic properties. They may control programmed cell death at different intracellular levels. Overexpression of HSPs may protect cells against apoptosis induced by Fas, TNF, ceramide, and cytotoxic drugs. It was shown that HSPs are involved in mitochondria dependent apoptotic pathways, preventing activation of caspases. HSP70 and HSP-90 interact with mutant p53, causing decrease in wild type p53, which is important regulator of cell cycle arrest/apoptosis.

### SUMMARY

In one aspect, the invention provides a method (typically a cosmetic method) of reducing unwanted mammalian (preferably human) hair growth by applying to the skin a heat shock protein (HSP) inhibitor in an amount effective to reduce hair growth. The unwanted hair growth may be undesirable from a cosmetic standpoint or may result, for example, from a disease or an abnormal condition (e.g., hirsutism).

HSP inhibitors include compounds that specifically inhibit the activity of one or more hair follicle HSPs by strongly interacting with the HSP(s); compounds that reduce the levels and/or expression of one or more HSPs in hair follicles; and/or compounds that reduce the expression of one or more HSP mRNA's in hair follicles. “Strongly interacts” means the compound binds or preferentially binds the HSP(s).

Typically, in practicing the aforementioned method, the inhibitor will be included in a topical composition along with a dermatologically or cosmetically acceptable vehicle. Accordingly, the present invention also relates to topical compositions comprising a dermatologically or cosmetically acceptable vehicle and an HSP inhibitor.

In addition, the present invention relates to the use of an HSP inhibitor for the manufacture of a therapeutic topical composition for reducing hair growth.

Specific compounds include both the compound itself and pharmacologically acceptable salts of the compound.

Other features and advantages of the invention may be apparent from the detailed description and from the claims.

### BRIEF DESCRIPTION OF DRAWINGS

The FIGURE provides microphotographs of hair follicles showing the localization of HSPs.

### DETAILED DESCRIPTION

A preferred composition includes an HSP inhibitor in a cosmetically and/or dermatologically acceptable vehicle. The composition may be a solid, semi-solid, or liquid. The composition may be, for example, a cosmetic and dermatologic product in the form of an, for example, ointment, lotion, foam, cream, gel, or solution. The composition may also be in the form of a shaving preparation or an aftershave. The vehicle itself can be inert or it can possess cosmetic, physiological and/or pharmaceutical benefits of its own.

Examples of known HSP inhibitors are provided in Table 1.

TABLE 1

| Name of Inhibitor | Chemical Name | Function | References |
|---|---|---|---|
| Geldanamycin | 2-Azabicyclo[16.3.1]docosa-4,6,10,18,21- | Inhibitor of | Stebbins, C. et al., Cell (1997), |
| | pentaene-3,20,22-trione, 9,13-dihydroxy-8,14,19- | HSP-90 | 89,239-250. |
| | trimethoxy-4,10,12,16-tetramethyl-, 9-carbamate | activity | Roe, S. Mark et al., Journal of |
| | (8CI); | | Medicinal Chemistry (1999), |
| | 2-Azabicyclo[16.3.1]docosane, geldanamycin | | 42, 260-266. |
| | deriv.; | | |
| | 2-Azabicyclo[16.3.1]docosa-4,6,10,18,21- | | |
| | pentaene-3,20,22-trione, 9-[(aminocarbonyl)oxy]- | | |
| | 13-hydroxy-8,14,19-trimethoxy-4,10,12,16- | | |
| | tetramethyl-, [8S- | | |
| | (4E,6Z,8R*,9R*,10E,12R*,13S*,14R*,16S*)]-; | | |
| | NSC 122750; NSC 212518; | | |
| | [8S-(4E,6Z,8R*,9R*,10E,12R*, | | |
| | 13S*,14R*,16S*)]-9-[(Aminocarbonyl)oxy]-13- | | |
| | hydroxy-8,14,19-trimethoxy-4,10,12,16- | | |
| | tetramethyl-2-azabicyclo[16.3.1]docosa- | | |
| | 4,6,10,18,21-pentaene-3,20,22-trione | | |
| 17-Allylamino,17- | 2-Azabicyclo[16.3.1] docosane, geldanamycin | Inhibitor of | Hostein et al., Cancer |
| demethoxygeldanamycin | deriv.; | HSP-90 | Research (2001), 61, 4003-4009. |
| | 17-Amino-17- demethoxygeldanamycin | activity | |
| KF25706, KF58333, | Oxime derivatives of radicicol; radicicol 6-oxime | Inhibitor of | Soga S et al., Cancer Res. |
| KF58332 | | HSP-90 | 1999 Jun. 15; 59(12): 2931-8. |
| | | activity | Shiotsu et al., Blood. 2000 Sep. 15; |
| | | | 96(6): 2284-91. |
| | | | Soga et al., 2003 |
| | | | Oct.; 3(5): 359-69. Review. |
| O-carbamoylmethyloxime | O-carbamoylmethyloxime derivatives of radicicol | Inhibitor of | Ikuina Y, et al., J Med Chem. |
| | | HSP-90 | 2003 Jun. 5; 46(12): 2534-41. |
| | | activity | |
| Benzo-1,3-dioxole | Benzo-1,3-dioxole | Inhibitor of | U.S. Pat. No. 6,613,780. |
| | | Heat Shock | |
| | | Factor | |
| | | activity, | |
| | | which leads | |
| | | to inhibition | |
| | | of HSP | |
| | | synthesis | |
| KNK 437 | 1-Pyrrolidinecarboxaldehyde, 3-(1,3- | Inhibitors | Koishi et al., Clinical Cancer |
| | benzodioxol-5-ylmethylene)-2-oxo-(9CI) | of HSP | Research (2001), 7, 215-219. |
| | N-Formyl-3,4-methylenedioxy-benzylidine-γ- | synthesis | Yokota et al., Cancer Research |
| | butyrolactam | | (2000), 60(11), 2942-2948. |
| KNK423 | 3,4-Methylenedioxy-benzylidine-γ-butyrolactam | | Yokota et al., PCT WO/98- |
| | | | JP2829 19980625. |

The composition may include more than one HSP inhibitor. In addition, the composition may include one or more other types of hair growth reducing agents, such as those described in U.S. Pat. No. 4,885,289; U.S. Pat. No. 4,720,489; U.S. Pat. No. 5,132,293; U.S. Pat. 5,096,911; U.S. Pat. No. 5,095,007; U.S. Pat. No. 5,143,925; U.S. Pat. No. 5,328,686; U.S. Pat. No. 5,440,090; U.S. Pat. No. 5,364,885; U.S. Pat. No. 5,411,991; U.S. Pat. No. 5,648,394; U.S. Pat. No. 5,468,476; U.S. Pat. No. 5,475,763; U.S. Pat. No. 5,554,608; U.S. Pat. No. 5,674,477; U.S. Pat. No. 5,728,736; U.S. Pat. 5,652,273; WO 94/27586; WO 94/27563; and WO 98/03149, all of which are incorporated herein by reference.

The concentration of the HSP inhibitor in the composition may be varied over a wide range up to a saturated solution, preferably from 0.1% to 30% by weight or even more; the reduction of hair growth increases as the amount of inhibitor applied increases per unit area of skin. The maximum amount effectively applied is limited only by the rate at which the inhibitor penetrates the skin. The effective amounts may range, for example, from 10 to 3000 micrograms or more per square centimeter of skin.

The vehicle can be inert or can possess cosmetic, physiological and/or pharmaceutical benefits of its own. Vehicles can be formulated with liquid or solid emollients, solvents, thickeners, humectants and/or powders. Emollients include stearyl alcohol, mink oil, cetyl alcohol, oleyl alcohol, isopropyl laurate, polyethylene glycol, petroleum jelly, palmitic acid, oleic acid, and myristyl myristate. Solvents include ethyl alcohol, isopropanol, acetone, diethylene glycol, ethylene glycol, dimethyl sulfoxide, and dimethyl formamide.

The composition optionally can include components that enhance the penetration of the inhibitor into the skin and/or to the site of action. Examples of penetration enhancers include urea, polyoxyethylene ethers (e.g., Brij-30 and Laureth-4), 3-hydroxy-3,7,11-trimethyl-1,6,10-dodecatriene, terpenes, cis-fatty acids (e.g., oleic acid, palmitoleic acid), acetone, laurocapram, dimethylsulfoxide, 2-pyrrolidone, oleyl alcohol, glyceryl-3-stearate, propan-2-ol, myristic acid isopropyl ester, cholesterol, and propylene glycol. A penetration enhancer can be added, for example, at concentrations of 0.1% to 20% or 0.5% to 5% by weight.

The composition also can be formulated to provide a reservoir within or on the surface of the skin to provide for a continual slow release of the inhibitor. The composition also may be formulated to evaporate slowly from the skin, allowing the inhibitor extra time to penetrate the skin.

A cream-based topical composition containing a HSP inhibitor is prepared by mixing together water and all water soluble components in a mixing vessel-A. The pH is adjusted in a desired range from about 3.5 to 8.0. In order to achieve complete dissolution of ingredients the vessel temperature may be raised to up to 45° C. The selection of pH and temperature will depend on the stability of the HSP inhibitor. The oil soluble components, except for the preservative and fragrance components, are mixed together in another container (B) and heated to up to 70° C. to melt and mix the components. The heated contents of vessel B are poured into the water phase (container A) with brisk stirring. Mixing is continued for about 20 minutes. The preservative components are added at temperature of about 40° C. Stirring is continued until the temperature reaches about 25° C. to yield a soft cream with a viscosity of 8,000-12,000 cps, or a desired viscosity. The fragrance components are added at about 25° C. -30° C. while the contents are still being mixed and the viscosity has not yet built up to the desired range. If it is desired to increase the viscosity of the resulting emulsion, shear can be applied using a conventional homogenizer, for example a Silverson L4R homogenizer with a square hole high sheer screen. The topical composition can be fabricated by including the active agent in the water phase during the aforedescribed formulation preparation or can be added after the formulation (vehicle) preparation has been completed. The active agent can also be added during any step of the vehicle preparation. The components of the cream formulations are described in the examples below.

### EXAMPLE #1

### Cream

| INCI Name | w/w (%) |
|---|---|
| DI Water | 61.00-75.00 |
| HSP Inhibitor^{a} | 1.00-15.00 |
| Mineral oil | 1.90 |
| Glyceryl stearate | 3.60 |
| PEG 100 Stearate | 3.48 |
| Cetearyl Alcohol | 2.59 |
| Ceteareth-20 | 2.13 |
| Dimethicone, 100 ct | 0.48 |
| Lipidure PMB^{b} | 3.00 |
| Advanced Moisture Complex^{c} | 5.00 |
| Stearyl alcohol | 1.42 |
| Preservative, fragrance and color pigment | qs |
| Total | 100.00 |

^{a}An HSP inhibitor can be selected, for example, from the list provided in Table 1.
^{b}Polyquartinium-51 (Collaborative Labs, NY).
^{c}Glycerin, water, sodium PCA, urea, trehalose, polyqauternium-51, and sodium hyaluronate (Collaborative Labs, NY).

### EXAMPLE #2

### Cream

| INCI Name | w/w (%) |
|---|---|
| HSP inhibitor^{a} | 0.5-15.00 |
| Glycerol (Glycerin) | 0-5 |
| Isoceteth-20 | 3-7 |
| Glyceryl isostearate | 1.5-5 |
| Dicaprylyl ether | 3-15 |
| Glyceryl triacetate (triacetin) | 0.5-10 |
| Preservative, fragrance and color pigment | q.s. |
| Water | q.s. to 100.00 |

^{a}An HSP inhibitor can be selected, for example, from the list provided in Table 1.

### EXAMPLE #3

### Cream

| INCI Name | w/w (%) |
|---|---|
| HSP inhibitor^{a} | 0.5-15.00 |
| Glycerol (Glycerin) | 0-5 |
| Isoceteth-20 | 3-7 |
| Glyceryl isostearate | 1.5-5 |
| Dicaprylyl ether | 3-15 |
| 1-dodecyl-2-pyrrolidanone | 0.5-10% |
| Preservative, fragrance and color | q.s. |
| Water | to 100.00 |

^{a}An HSP inhibitor can be selected, for example, from the list provided in Table 1.

### EXAMPLE #4

### Cream

| INCI Name | w/w (%) |
|---|---|
| Water | 70 |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| 1-Dodecyl-2-pyrrolidanone | 1-10 |
| Total | 100.00 |

An HSP inhibitor is added to the example 4 formulation and mixed until solubilized. A HSP inhibitor can be selected, for example, from the list provided in Table 1.

### EXAMPLE #5

### Cream

| INCI Name | w/w (%) |
|---|---|
| Water | 70-80 |
| Glyceryl Stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Monocaprylate/caprate (Estol 3601, Uniquema, NJ) | 1-10 |
| Total | 100.00 |

An HSP inhibitor is added to the example 4 formulation and mixed until solubilized. A HSP inhibitor can be selected, for example, from the list provided in Table 1.

### EXAMPLE #6

### Cream

| INCI Name | w/w (%) |
|---|---|
| Water | 70-80 |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| cis Fatty acids | 1-10 |
| Total | 100.00 |

An HSP inhibitor is added to the example 4 formulation and mixed until solubilized. An HSP inhibitor can be selected, for example, from the list provided in Table 1.

### EXAMPLE #7

### Cream

| INCI Name | w/w (%) |
|---|---|
| Water | 70-80% |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Terpene(s) | 1-10 |
| Total | 100.00 |

An HSP inhibitor is added to the example 4 formulation and mixed until solubilized. An HSP inhibitor can be selected, for example, from the list provided in Table 1.

### EXAMPLE #8

### Cream

| INCI Name | w/w (%) |
|---|---|
| Water | 70-80% |
| Glyceryl stearate | 4 |
| PEG-100 | 4 |
| Cetearyl alcohol | 3 |
| Ceteareth-20 | 2.5 |
| Mineral oil | 2 |
| Stearyl alcohol | 2 |
| Dimethicone | 0.5 |
| Preservatives | 0.43 |
| Polyoxyethylene sorbitans (tween) | 1-10 |
| Total | 100.00 |

An HSP inhibitor is added to the example 4 formulation and mixed until solubilized. An HSP inhibitor can be selected, for example, from the list provided in Table-1.

A hydroalcoholic formulation containing an HSP inhibitor is prepared by mixing the formulation components in a mixing vessel. The pH of the formulation is adjusted to a desired value in the range of 3.5-8.0. The pH adjustment can also be made to cause complete dissolution of the formulation ingredients. In addition, heating can be applied to up to 45° C., or even up to 70° C. depending on the stability of the active in order to achieve dissolution of the formulation ingredients. Several hydroalcoholic formulations are listed below.

### EXAMPLE #9

### Hydro-Alcoholic

| INCI Name | w/w (%) |
|---|---|
| Water | 48.00-62.50 |
| HSP inhibitor^{a} | 0.5-15.00 |
| Ethanol | 16.00 |
| Propylene glycol | 5.00 |
| Dipropylene glycol | 5.00 |
| Benzyl alcohol | 400 |
| Propylene carbonate | 2.00 |
| Captex-300^{b} | 5.00 |
| Total | 100.00 |

^{a}An HSP inhibitor can be selected, for example, from the list provided in Table 1.
^{b}Caprylic/capric triglyceride (Abitec Corp., OH).

### EXAMPLE #10

### Hydro-Alcoholic

| INCI Name | w/w (%) |
|---|---|
| Water | 53.00-67.9 |
| HSP inhibitor^{a} | 0.1-15.00 |
| Ethanol | 16.00 |
| Propylene glycol | 5.00 |
| Dipropylene glycol dimethyl ether | 5.00 |
| Benzyl alcohol | 4.00 |
| Propylene carbonate | 2.00 |
| Total | 100.00 |

^{a}An HSP inhibitor can be selected, for example, from the list provided in Table 1.

### EXAMPLE #11

### Hydro-Alcoholic

| INCI Name | w/w (%) |
|---|---|
| Ethanol (alcohol) | 80 |
| Water | 17.5 |
| Propylene glycol dipelargonate | 2.0 |
| Propylene glycol | 0.5 |
| Total | 100.00 |

An HSP inhibitor is added to the formulation and mixed until solubilized. An HSP inhibitor can be selected, for example, from the list provided in Table 1.

The composition should be applied topically to a selected area of the body from which it is desired to reduce hair growth. For example, the composition can be applied to the face, particularly to the beard area of the face, i.e., the cheek, neck, upper lip, and chin. The composition also may be used as an adjunct to, for example, shaving or mechanical epilation.

The composition can also be applied to the legs, arms, torso or armpits. The composition is particularly suitable for reducing the growth of unwanted hair in women having hirsutism or other conditions. In humans, the composition should be applied once or twice a day, or even more frequently, to achieve a perceived reduction in hair growth. Perception of reduced hair growth could occur as early as 24 hours or 48 hours (for instance, between normal shaving intervals) following use or could take up to, for example, three months. Reduction in hair growth is demonstrated when, for example, the rate of hair growth is slowed, the need for removal is reduced, the subject perceives less hair on the treated site, or quantitatively, when the weight of hair removed (i.e., hair mass) is reduced.

### Human Hair Follicle Growth Assay

Human skin was obtained from a plastic surgeon as a by-product of face-lift procedures. The skin samples generally consisted of haired and non-haired regions taken from the area of the face. Immediately after removal, the skin was placed in Williams E medium containing antibiotics, and kept refrigerated. The Williams E medium was commercially obtained (Life Technologies, Gaithersburg, Md.), and has been formulated with essential nutrients for maintaining viability of hair follicle in an in-vitro environment.

Human hair follicles in growth phase (anagen) were isolated from face-lift tissue under a dissecting scope using a scalpel and watchmakers forceps. The skin was sliced into thin strips exposing 2-3 rows of follicles that could readily be dissected. Follicles were placed into 0.5 ml Williams E medium supplemented with 2 mM L-glutamine, 10 ug/ml insulin, 10 ng/ml hydrocortisone, 100 units penicillin, 0.1 mg/ml streptomycin and 0.25 pg/ml amphotericin B. The follicles were incubated in 24 well plates (1 follicle/well) at 37° C. in an atmosphere of 5% CO₂ and 95% air. Hair follicle images were taken in the 24-well plates under the dissecting scope under a power of 20×. Hair follicle lengths were measured on day 0 (day follicles were placed in culture) and again on day 6-7. In this system follicles appear to fully differentiate into a hair fiber and increase in length at a rate similar to the human, in vivo, rate of about 0.3 mm/day. For testing inhibitors of heat shock proteins, the inhibitors or anti-HSP antibody were included in the culture medium from time 0 and remained in the medium throughout the course of the experiment.

### Immunohistochemistry Assay

Eight-micron cryosections through hair follicles or quick frozen skin biopsy were prepared and fixed in acetone for 10 minutes at −20° C. For the immunodetection of HSP-27, HSP70 or HSP-90, the tyramide-amplification method was used. Briefly, after blocking endogenous peroxidase and non-specific avidin/biotin binding (Avidin/Biotin blocking kit, Vector Lab), sections were incubated in TNB buffer (0.1 M Tris-HCl, pH 7.5, 0.15 M NaCl, and 0.5% Blocking Reagent, Perkin Elmer, Boston, Mass.) for 30 minutes. Next, mouse monoclonal antibody against human HSP-27, HSP70 (Calbiochem) or rabbit polyclonal antibody against human HSP-90 (Santa Cruz Biotechnology) were applied overnight (1:500 and 1:1000 respectively), followed by application of the biotinylated goat anti-mouse or goat anti-rabbit antiserum, diluted in TNB blocking buffer (Perkin Elmer, Boston, Mass., 1:200, 30 min). Subsequently, sections were incubated in streptavidin-horse radish peroxidase (1:100 in TNB, 30 min). Three washes with TNT buffer (0.1 M Tris-HCl, pH 7.6, 0.15M NaCl, 0.05% Tween) were followed by a 10 min application of TRITC-tyramide (1:50 in Amplification Diluent, Perkin Elmer, Boston, Mass.). Next, sections were counterstained with Hoechst 33342 for identification of cell nuclei, and mounted using VectaShield (Vector Laboratories).

All sections were examined under a Olympus BX 60 fluorescent microscope and photodocumented with the help of a digital image analysis system (CoolSnap™ cooled CD camera, Alpha Innotech).

### Results

Using immunohistochemical methodology, the presence of HSP-27, HSP-70 and HSP-90 was demonstrated in human hair follicles in vitro (see FIG. 1). HSP-27 and HSP-70 were found in the follicular epithelium and mesenchyme cells in well defined compartments, such as the outer root sheath and the dermal papilla cells of the follicle. HSP-90 on the other hand though more broadly expressed in the hair follicle epithelium was absent in dermal papilla cells of the mesenchyme origin. This immunohistochemical methodology can be used to select an agent that specifically reduces the levels and/or expression of the HSP-27 and HSP-70 or HSP-90.

Additional immunohistochemical assays were performed to determine changes in HSP expression and the specificity of agents binding to a HSP. To examine the role of HSP in human hair follicle development, endogenous HSP-27 was neutralized by adding into the culture medium an anti-HSP-27 antibody. Isolated human hair follicles were cultured in supplemented William's medium in the presence of anti-HSP-27 antibody at a concentration 1 μg/ml. Forty-eight hours later, immunohistochemical analysis of HSP-27 was performed to determine the expression of HSP-27 in the follicles. Analysis of control hair follicles revealed a strong expression HSP-27 in the follicular epithelium and mesenchyme cells. In contrast, the hair follicles treated with anti-HSP-27 antibody showed complete inhibition of HSP-27 expression in the proximal hair follicle compartments. Isolated cells in the distal outer root sheath remained HSP-27 positive. Results of human hair follicles treated with the monoclonal anti-HSP-27 antibody demonstrate:
(1) A significant antibody binding to the HSP-27 protein as determined by the immunohistochemical analysis;
(2) The anti-HSP-27 antibody inhibited the activity and expression of endogenous HSP-27 as a result of this strong binding to the protein;
(3) An increased incidence of catagen development (67% for the anti-HSP-27 antibody treated follicles versus 16% in the control, data shown in Table 2);
(4) A significant reduction of hair fiber growth (data shown in Table 2); and

(5) A method for selecting additional HSP inhibitors.

TABLE 2
Reduction of human hair growth and catagen induction by treatment with
anti-HSP-27 antibody

| | | Length | % | % |
|---|---|---|---|---|
| Compound | Dose | Increase (mm) | Reduction^{a} | Catagen |
| Control | — | 1.55 ± .11 | | 16 |
| anti-HSP-27 Antibody | 1 μg/ml | 0.89 ± .22 | 42 | 67 |

^{a}Hair growth was determined by subtracting total hair follicle length on day 0 from total hair follicle length on day 5.
% Reduction = 100-(hair growth for inhibitor treated follicles/hair growth for control) × 100.
% Catagen = (catagen hair follicle number/total hair follicle number) × 100.

A dose-dependent reduction of human hair follicle growth was seen with the HSP-90-specific inhibitor geldanamycin (Table 3). A strong, over 50% reduction of hair growth at the submicromolar dose of the inhibitor demonstrates the dependence of hair growth on optimally functioning HSP activity.

TABLE 3
Reduction of human hair growth by an HSP-90 inhibitor

| Compound | Dose | Length Increase (mm) | % Reduction^{a} |
|---|---|---|---|
| Control | — | 1.16 ± 0.2 | 0 |
| Geldanamycin | 0.1 μM | 0.55 ± 0.13 | 52 |
| Geldanamycin | 1 μM | 0.30 ± 0.08 | 74 |

^{a}Hair growth was determined by subtracting total hair follicle length on day 0 from total hair follicle length on day 6. % Inhibition = 100-(hair growth for inhibitor treated follicles/hair growth for control) × 100.

Table 4 shows the dose-dependent reduction of human hair follicle growth by KNK 437, a benzylidene lactam compound. The compound KNK 437 is known to inhibit the induction, and thus expression, of HSPs at the mRNA level.

Table 4: Reduction of human hair growth by KNK 437.

TABLE 4
Reduction of human hair growth by KNK 437.

| | | Length Increase | |
|---|---|---|---|
| Compound | Dose | (mm) | % Reduction^{a} |
| Control | — | 1.72 ± 0.17 | 0 |
| KNK 437 | 10 μM | 0.75 ± 0.13 | 57 |
| | | | (p < 0.0001) |
| KNK 437 | 50 μM | 0.35 ± 0.12 | 80 |
| | | | (p < 0.000005) |

^{a}Hair growth was determined by subtracting total hair follicle length on day 0 from total hair follicle length on day 5. % Inhibition = 100-(hair growth for inhibitor treated follicles/hair growth for control) × 100. % Catagen = (catagen hair follicle number/total hair follicle number) × 100.

Other embodiments are within the claims.

1. A method of reducing mammalian hair growth which comprises
selecting an area of skin from which reduced hair growth is desired; and
applying to said area of skin a dermatologically acceptable composition comprising an a heat shock protein inhibitor in an amount effective to reduce hair growth.

2. The method of claim 1, wherein said inhibitor is geldanamycin.

3. The method of claim 1, wherein said inhibitor is 17-allylamino,17-demethoxygeldanamycin.

4. The method of claim 1, wherein said inhibitor is KF25706.

5. The method of claim 1, wherein inhibitor is KF58333.

6. The method of claim 1, wherein said inhibitor is KF58332.

7. The method of claim 1, wherein said inhibitor is O-carbamoylmethyloxime.

8. The method of claim 1, wherein said HSP inhibitor is geldanamycin benzo-1,3-dioxole.

9. The method of claim 1, wherein said inhibitor is KNF437.

10. The method of claim 1, wherein said inhibitor is KNK423.

11. The method of claim 1, wherein said inhibitor is a compound that can inhibit the activity of one or more hair follicle heat shock proteins.

12. The method of claim 1, wherein said inhibitor is a compound that can bind to one or more hair follicle heat shock proteins and inhibit the function of the bound protein.

13. The method of claim 1, wherein said inhibitor is a compound that can reduce levels and/or expression of one or more heat shock proteins in hair follicles.

14. The method of claim 1, wherein said inhibitor is a compound that can reduce the expression of one or more heat shock protein mRNA's in hair follicles.

15. The method of claim 1, wherein the concentration of said inhibitor in said composition is between 0.1% and 30%.

16. The method of claim 1, wherein said inhibitor is applied to the skin in an amount of from 10 to 3000 micrograms of said inhibitor per square centimeter of skin.

17. The method of claim 1, wherein said mammal is a human.

18. The method of claim 17, wherein said area of skin is on the face of a human.

19. The method of claim 17, wherein the composition is applied to the area of skin in conjunction with shaving.

20. The method of claim 17, wherein said area of skin is on a leg of the human.

21. The method of claim 17, wherein said area of skin is on an arm of the human.

22. The method of claim 17, wherein said area of skin is in an armpit of the human.

23. The method of claim 17, wherein said area of skin is on the torso of the human.

24. The method of claim 1, wherein the composition is applied to an area of skin of a woman with hirsutism.

25. The method of claim 1, wherein said hair growth comprises androgen stimulated hair growth.

26. The method of claim 1, wherein the composition further includes a second component that also causes a reduction in hair growth.
